(19) United States
(12) Patent Application Publication
(10) Pub. No.: US 2016/0202253 A1
(43) Pub. Date: Jul. 14, 2016

(54) DETECTION OF BIOLOGICAL MOLECULES USING SURFACE PLASMON FIELD ENHANCED FLUORESCENCE SPECTROSCOPY (SPFS) COMBINED WITH ISOTACHOPHORESIS (ITP)

(71) Applicant: KONICA MINOLTA LABORATORY U.S.A., INC., San Mateo, CA (US)

(72) Inventor: Noriaki Yamamoto, Tokyo (JP)

(73) Assignee: KONICA MINOLTA LABORATORY U.S.A., INC., San Mateo, CA (US)

(21) Appl. No.: 14/995,112

(22) Filed: Jan. 13, 2016

Related U.S. Application Data
(60) Provisional application No. 62/102,673, filed on Jan. 13, 2015.

(51) Int. Cl. G01N 33/543; G01N 33/553; G01N 21/64; B01L 3/00

(52) U.S. Cl. CPC G01N 33/54366; B01L 3/502715; G01N 33/553; G01N 21/648; G01N 21/6428; B01L 2300/0867; B01L 2300/0654; B01L 2300/12; B01L 2300/16; G01N 2021/6417

(57) ABSTRACT

A method for detecting biological molecules that combines surface plasmon field-enhanced fluorescence spectroscopy (SPFS) and Isotachophoresis (ITP). An ITP setup, including a TE reservoir, an LE reservoir, and a fluid channel connecting the two, is equipped on the SPFS sensor, such that the solution in the fluid channel passes the SPFS sensor surface between the TE reservoir and the LE reservoir. Target analytes and fluorescent labeled probes loaded into the TE reservoir are focused in a region of fluid channel upstream from the SPFS sensor region, and they are reacted. The focused sample travels downstream to reach the SPFS sensor region, and the analyte-probe complexes are captured by capture molecules immobilized on the sensor surface. After the focused sample completely passes through the SPFS sensor region, captured fluorescent molecules on the sensor surface are detected using the SPFS mechanism.

### BACKGROUND OF THE INVENTION

1. Field of the Invention

This invention relates to a method that uses surface plasmon field enhanced fluorescence spectroscopy (SPFS) and isotachophoresis (ITP) to achieve ultra-rapid and highly-sensitive biological molecules detection.

2. Description of Related Art

Surface plasmon field-enhanced fluorescence spectroscopy (SPFS) is a known biosensing technology. See T. Liebermann, W. Knoll, Surface-plasmon field-enhanced fluorescence spectroscopy, Colloids and Surfaces A: Physicochem. Eng. Aspects 171 (2000) 115-130 (“Liebermann 2000”); Wolfgang Knoir, Fang Yu, Thomas Neumann, Lifang Niu, and Evelyne L. Schmid, Principles And Applications Of Surface Plasmon Field-Enhanced Fluorescence Techniques, in Topics in Fluorescence Spectroscopy, Volume 8: Radiative Decay Engineering, Edited by Geddes and Lakowicz, Springer Science+Business Media, Inc., New York, 2005, p. 305-332. These references are incorporated by reference in their entireties to show the principle and setup of SPFS biosensors in general. SPFS offers high-sensitivity detection through advanced sensing technology.

FIG. 1A of this application, taken from FIG. 5 of the Liebermann 2000 paper, illustrates the setup of an SPFS system. FIG. 1B of this application, taken from FIG. 6(a) of the same paper, illustrates the structure of the prism and flow cell used in the SPFS system. The basic concept of SPFS is described below with reference to FIGS. 1, 1A and 1B. An SPFS biosensor includes a thin metal film on a glass or plastic prism. The metal may be, for example, gold, silver, aluminum, etc. A capture molecule is immobilized on the surface of the metal film. A biological sample is applied on the metal film. When an incident light of a certain wavelength is irradiated on the prism at a certain angle, a strong electrical field is generated at the surface of the metal film. Because of quenching from the metal film, the best place for fluorescence excitation is in the region about a couple of tens to hundreds nm above the surface. In a typical device, the quenching region is within about 0-5 nm from the metal surface, and the enhanced region is about 10-200 nm from the surface. If a fluorescent label is trapped in this enhanced region, strong fluorescent signal is generated.

SPFS biosensors are based on fluorescence detection. In conventional SPFS biosensors, in addition to first antibodies that are immobilized on the thin metal film, fluorescent labeled second antibodies are generally used for protein detection. This is schematically illustrated in FIG. 1. The first antibodies 101 are immobilized on the thin metal film. The target 102 (i.e. substance to be detected, such as a protein) is added to the biosensor and captured on the immobilized first antibodies. Then, the fluorescent labeled second antibodies 103 are added to the biosensor and they bind to the target. The first antibody 101, the target 102 and the second antibody 103 form a structure such that the fluorescent label 103F on the second antibody is located in the region of enhanced electric field above the thin metal film, and a strong fluorescent signal is generated. For unbound second antibodies or those that form non-specific binding, their fluorescent labels tend to be located outside of the enhanced region, either in the metal quenching region or farther away from the surface, so they are not excited. The biosensor can be washed before the detection result is obtained. These multiple steps make the biosensor more complicated to use and the turnaround time long.

PCT application WO 2011155435 A1, Near field-enhanced fluorescence sensor chip, also describes surface plasmon field enhanced fluorescence spectroscopy.

Isotachophoresis (ITP) is an electrophoresis technique that uses two buffers including a high mobility leading electrolyte (LE) and a low-mobility trailing electrolyte (TE). In peak-mode ITP, sample species bracketed by the LE and TE focus into a narrow TE-to-LE interface. Due to the high concentration of sample species in a small volume at the interface, high efficiency (rapid) molecular-molecular interaction can occur.

An ultra-rapid nucleic acid detection technology using ITP is described in Rapid Detection of Urinary Tract Infections Using Isotachophoresis and Molecular Beacons, M. Bercovici et al., Analytical Chemistry 2011, 83, 4110-4117 (“Bercovici et al. Analytical Chemistry 2011”). This method accelerates DNA hybridization by using ITP. FIG. 1 of this article, reproduced as FIG. 2 of the instant disclosure, shows the principle of detection. The article describes: “FIG. 1a schematically presents the principles of the assay. ITP uses a discontinuous buffer system consisting of LE and TE, which are typically chosen to have respectively higher and lower electrophoretic mobility than the analytes of interest. Both sample and molecular beacons are initially mixed with the TE. When an electric field is applied, all species with mobility higher than that of the TE electromigrate into the channel. Other species (including ones with lower mobility, neutral or positively charged) remain in or near the sample reservoir. Focusing occurs within an electric field gradient at interface between the LE and TE, as sample ions cannot overspeed the LE zone but overspeed TE ions.” (Id., p. 4111, left column.) “FIG. 1. (a) Schematic showing simultaneous isotachophoretic extraction, focusing, hybridization (with molecular beacons), and detection of 16S rRNA bound to a molecular beacon. Hybridization of the molecular beacon to 16S rRNA causes a spatial separation of its fluorophore and quencher pair resulting in a strong and sequence-specific increase in fluorescent signal. (b) Raw experimental image showing fluorescence intensity of molecular beacons hybridized to synthetic oligonucleotides using ITP. (c) Detection of oligonucleotides having the same sequence as the target segment of 16S rRNA. Each curve presents the fluorescence intensity in time, as recorded by a point detector at a fixed location in the channel (curves are shifted in time for convenient visualization). 100 pM of molecular beacons and varying concentrations of targets were mixed in the trailing electrolyte reservoir. The total migration (and hybridization) time from the on-chip reservoir to the detector was less than a minute.” (Id., p. 4111, right column.) A setup for the on-chip ITP assay using a microfluidic chip is shown in FIGS. 2A and 2B of the instant disclosure, reproduced from FIGS. 2 and 3(a) of the above article.

Han, C. M., Katilius, E., Santiago, J. G., “Increasing hybridization rate and sensitivity of DNA microarrays using isotachophoresis,” Lab on a Chip 2014 discloses a method to increase hybridization between immobilized DNA probe and free DNA by ITP.

### SUMMARY

For conventional SPFS, since the sample volume is usually larger than the volume of the sensor region, it takes time for the entire sample to react with the sensor region. So there is a need to confine the sample in the sensor surface area within a short time.

By using ITP technology, rapid sample confinement can be achieved; however, the short reaction time between the analyte and capture molecules on the SPFS sensor surface due to rapid movement of the sample at the TE/LE interface in ITP is a concern. Also, due to the characteristics of ITP which concentrate samples, non-specific signal increase may be a problem. Non-specific signals can be due to binding of other components in the biological sample (other than the analyte) to the fluorescent labeled probe and capture molecule in the SPFS sensor, or binding of labeled probe directly to the capture molecule.

Accordingly, the present invention is directed to a method that substantially obviates one or more of the problems due to limitations and disadvantages of the related art.

An object of this invention is to achieve ultra-rapid and highly-sensitive detection by combining ITP and SPFS.

To achieve these and/or other objects, as embodied and broadly described, the present invention provides a method for detecting a target analyte, which includes: using isotachophoresis (ITP) to concentrate a target analyte and a fluorescent labeled probe and allow them to form a complex, allowing the target analyte and fluorescent labeled probe complex to be captured by capture molecules on the sensor surface of a surface plasmon field enhanced fluorescence spectroscopy (SPFS), and detecting a fluorescent signal emitted by the captured fluorescent labeled probe by SPFS.

Other features include: The retention time of the concentrated sample on the SPFS sensor surface is extended. This may be done by controlling the applied voltage and/or increasing the sensor surface area size. The voltage control can be started when the concentrated sample reaches the SPFS sensor surface, and the timing is calculated by the sample's velocity in advance or is obtained by detecting fluorescent signal in the sample during test. Unbound fluorescent probe can be captured by a filter located in the ITP fluid channel upstream of the SPFS sensor. A TE buffer with strong wash effect can be used to wash the SPFS sensor and SPFS detection is conducted after washing.

Using techniques described herein, not only by concentrating the sample by ITP, but also by extending the time duration that the concentrated sample is located on the SPFS sensor surface, rapid and highly-sensitive sensing can be achieved. In this case, there is no need to use microfluidics based on the pump, which usually takes a long time.

Non-specific binding reducing mechanisms are employed to reduce non-specific binding.

Furthermore, because the fluorescent labeled probes are captured by the SPFS sensor surface, there is flexibility in the timing of signal detection, that is, there is no need to detect the signal at a fixed timing.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

FIGS. 1, 1A and 1B schematically illustrate the principle and setup of a conventional SPFS biosensor.

FIGS. 2, 2A and 2B schematically illustrate the principle of a biomolecule detection method using ITP and a setup for the on-chip ITP assay in a known method.

FIG. 3 schematically illustrates the principle of a biomolecule detection method combining SPFS and ITP according to an embodiment of the present invention.

FIGS. 4 and 5 schematically illustrate two methods for extending the concentrated sample retention time according to embodiments of the present invention.

FIG. 6 schematically illustrates a method and setup for reducing non-specific binding in the biomolecule detection method according to an embodiment of the present invention.

FIG. 7 illustrates an example of a voltage control sequence in a detection method using ITP equipped with a filter and SPFS sensor according to an embodiment of the present invention.

FIG. 8 schematically illustrates a DNA detection method employing DNAzyme amplification and separation mechanisms in combination with ITP and SPFS techniques according to an embodiment of the present invention.

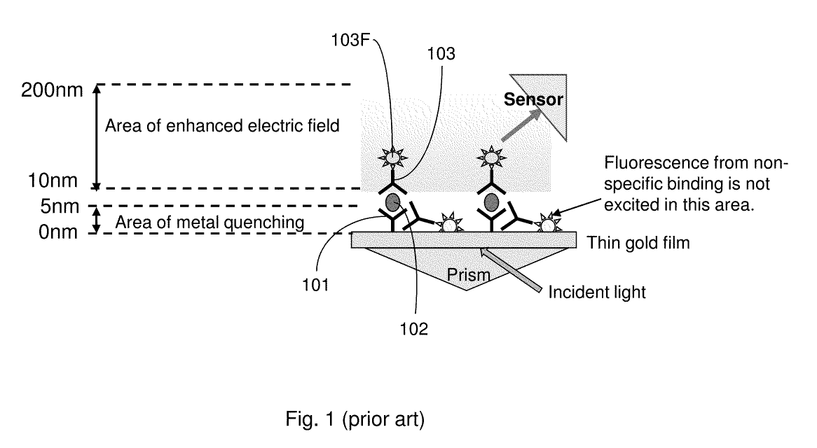

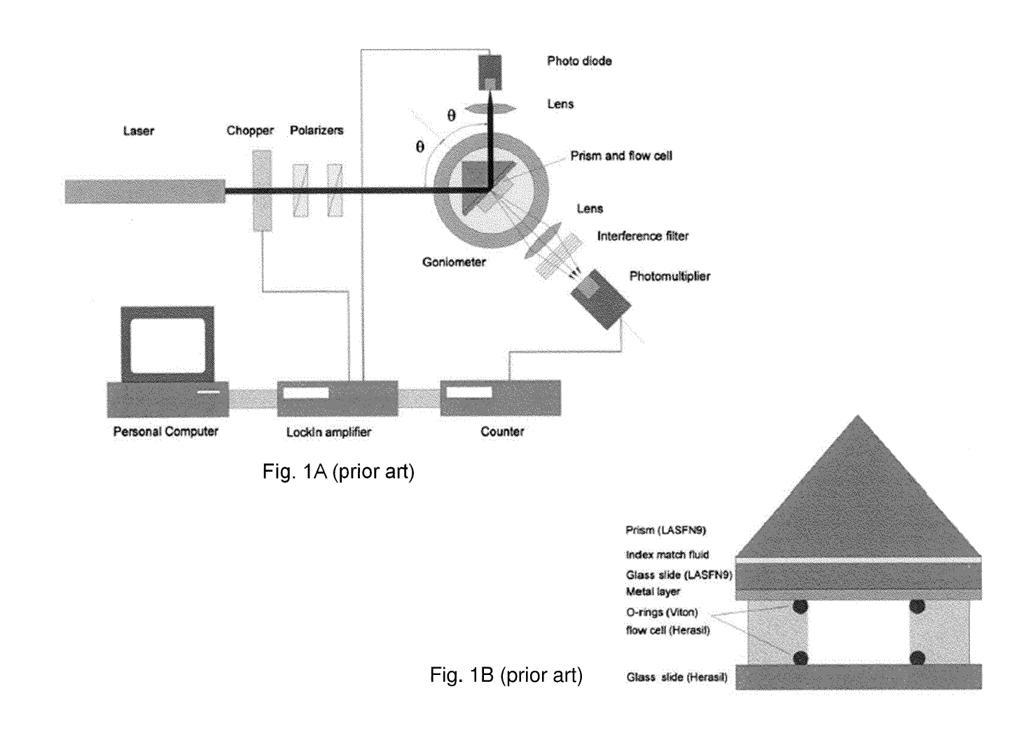

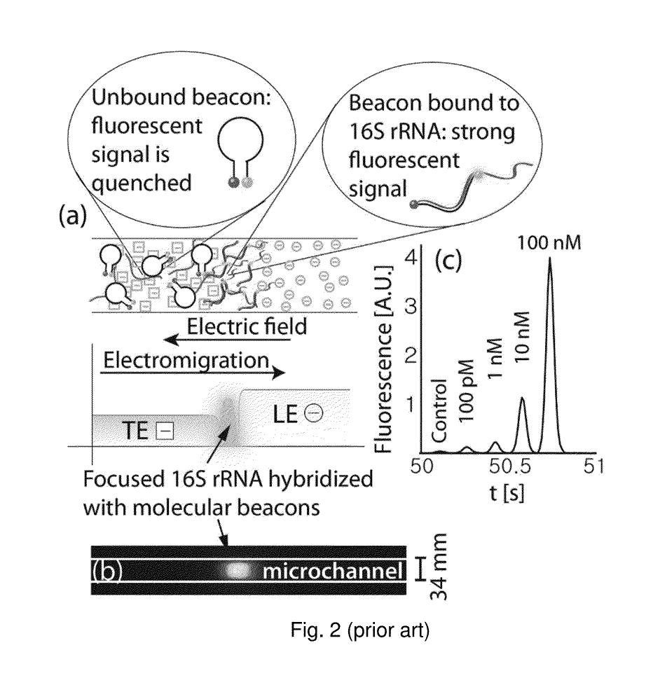

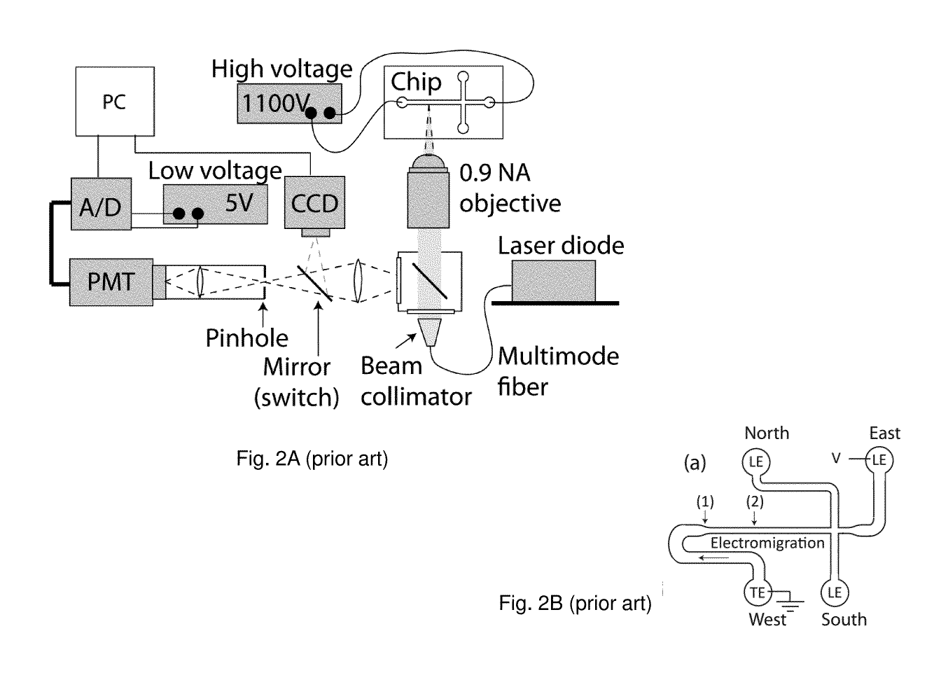

### DETAILED DESCRIPTION OF PREFERRED EMBODIMENTS

A novel combination of SPFS and ITP technologies is disclosed herein. The potential challenges caused by the combination, such as short reaction time and non-specific binding, can be overcome by using various techniques described below. To summarize, the potential problem of short reaction time is solved by extending the concentrated sample retention time on the SPFS sensor surface, specifically, (1) by controlling sample movement speed by voltage control (slow down, stop, reverse, etc.), and/or (2) by expanding the capture area of the SPFS sensor. The non-specific binding is reduced by (1) introducing a filter upstream from the SPFS sensor, and/or (2) using a special wash buffer.

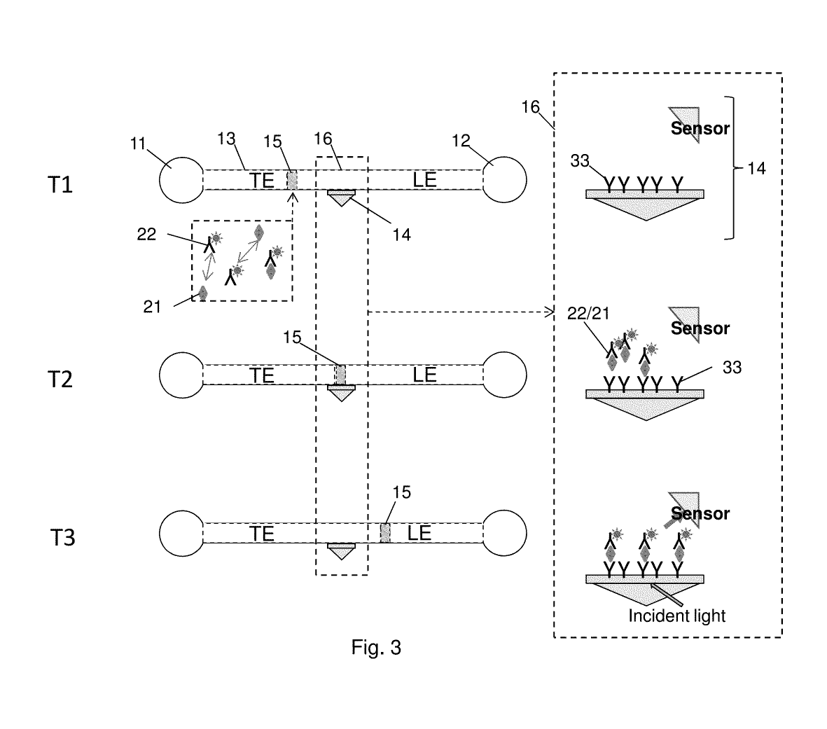

FIG. 3 schematically illustrates the principles of a method that combines SPFS and ITP according to an embodiment of the present invention. An ITP setup, including a TE reservoir 11, an LE reservoir 12, and a fluid channel 13 connecting the two, is equipped on the SPFS sensor 14, such that the solution in the fluid channel passes and contacts the SPFS sensor surface (i.e. the surface of the thin metal film on the prism) between the TE reservoir and the LE reservoir. In time period T1, target analytes 21 and fluorescent labeled probes 22 which have been loaded into the TE reservoir 11 are focused 15 in a region of the fluid channel upstream from the SPFS sensor region 16, and they are reacted (i.e. the target analyte binds to the probes). In time period T2, the focused sample 15 travels downstream to reach the SPFS sensor region 16, and the analyte-probe complexes are captured by capture molecules 33 immobilized on the sensor surface. In time period T3, after the focused sample 15 completely passes through the SPFS sensor region 16, captured fluorescent molecules 21/22 on the sensor surface 16 are detected using the SPFS mechanism, i.e. by irradiating an incident light on the SPFS sensor and detecting the output fluorescent signal. The mobility (μ) of the various components in the ITP system should satisfy μ_{LE}>μ_{target}, μ_{labeled probe}>μ_{TE}.

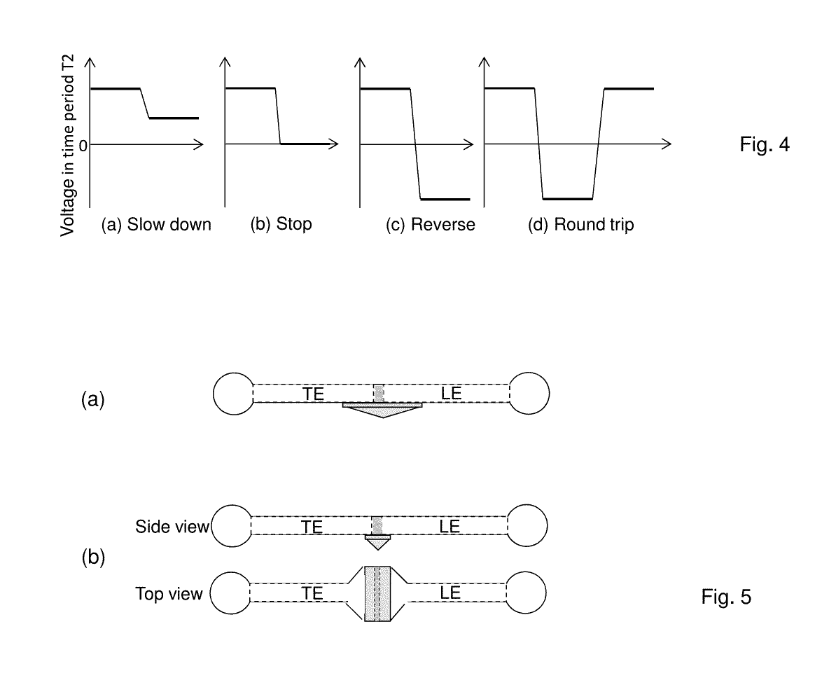

FIGS. 4 and 5 schematically illustrate two methods for extending the concentrated sample retention time, i.e. the time duration that the concentrated sample is located within the region 16 of the microchannel above the SPFS sensor surface.

The first method involves changing the voltage applied between the TE and LE reservoirs 11 and 12 in the ITP setup. As shown in FIG. 4, in time period T2, i.e. when the focused sample reaches the SPFS sensor region 16, voltage profile (a) which applies a reduced voltage level may be used to slow down the sample in the sensor region; voltage profile (b) where the voltage is reduced to zero may be used to stop the sample in the sensor region; voltage profile (c) which applies a voltage of a reversed polarity may be used to cause the sample to travel in the reverse direction in the sensor region; and voltage profile (d) which applies voltages of alternating polarities may be used to cause the sample to repeatedly travel back and forth in the sensor region. Combinations of the above voltage profiles can also be used. The voltage can be changed either in a gradual manner or in a discrete manner. A DC voltage is used in the above examples, but an AC voltage can be also used.

It should be noted that a lower voltage or a zero voltage causes the focused sample band to be diffused, which is not desirable; therefore, in determining the voltage control pattern, there is a tradeoff between extending the sample retention time and maintaining concentration of the sample.

The timing of when the concentrated sample will reach the sensor region can be calculated using expected sample migration speed (V_{ITP}=μ_{LE}*E_{LE}) in advance, and voltage variation control can be started at that time. Alternatively, the timing of when the concentrated sample reaches the sensor region can be detected by detecting the fluorescent molecules in the sample using the SPFS sensor during the test. As another alternative, a colored material which has a mobility μ_{color} satisfying (μ_{LE}>μ_{color}≧μ_{target}, μ_{labeled probe}) is mixed with the sample and used for position monitoring.

The second method for extending the concentrated sample retention time involves increasing the size of the SPFS sensor surface, as shown in FIG. 5. The SPFS sensor surface can be increased in the direction parallel to the travel direction of the sample solution (i.e. along the fluid channel) (FIG. 5(a)), or in the direction perpendicular to the travel direction (FIG. 5(b)), e.g. by aligning multiple capillaries. It is preferable that the width (i.e. the dimension in the sample travel direction) of the sensor surface area is larger than the width of sample band focused by ITP, so as to increase the time that the sample is located in the sensor region. It should be noted that if the sensor surface area is increased, the irradiation light and the prism of the SPFS device also need to be increased to detect the signals from the entire sensor surface area.

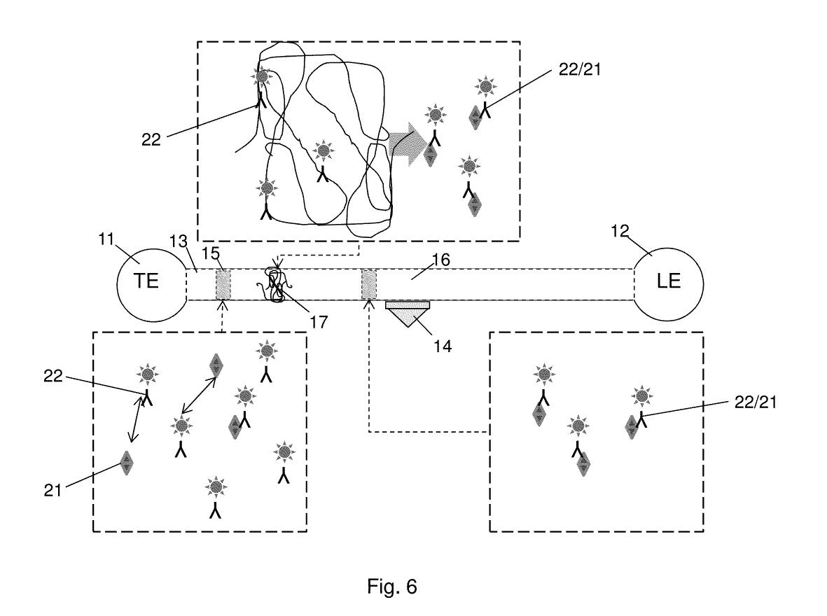

A method for reducing non-specific binding is illustrated in FIG. 6. A filter 17, located in the fluid channel 13 between the TE reservoir 11 and the SPFS sensor region 16, is used to capture unbound labeled probes 22, whereas the probe-analyte complex 22/21 passes through the filter. When the concentrated sample reaches the filter 17, the applied voltage is controlled (e.g., similar to the examples shown in FIG. 4) to cause the sample to slow down, stop, reverse, or travel back and forth, in order to increase the capture of the unbound labeled probe by the filter. It should be noted that the location of the filter 17 should allow the binding of the probe 22 and the analyte 21 to occur sufficiently before the sample reaches the filter.

Another method (not shown in the drawings) for reducing non-specific binding is to use a TE buffer that has a strong wash effect to wash off the non-specifically bound fluorescent molecules (labeled probed) from the SPFS sensor surface. Generally speaking, the requirements for the TE buffer are not very strict and it is not difficult to find appropriate wash buffers that will be suitable as the TE buffer. Examples of strong wash buffers that can be used as the TE buffer include surfactants such as TritonX-100, Tween 20, etc.

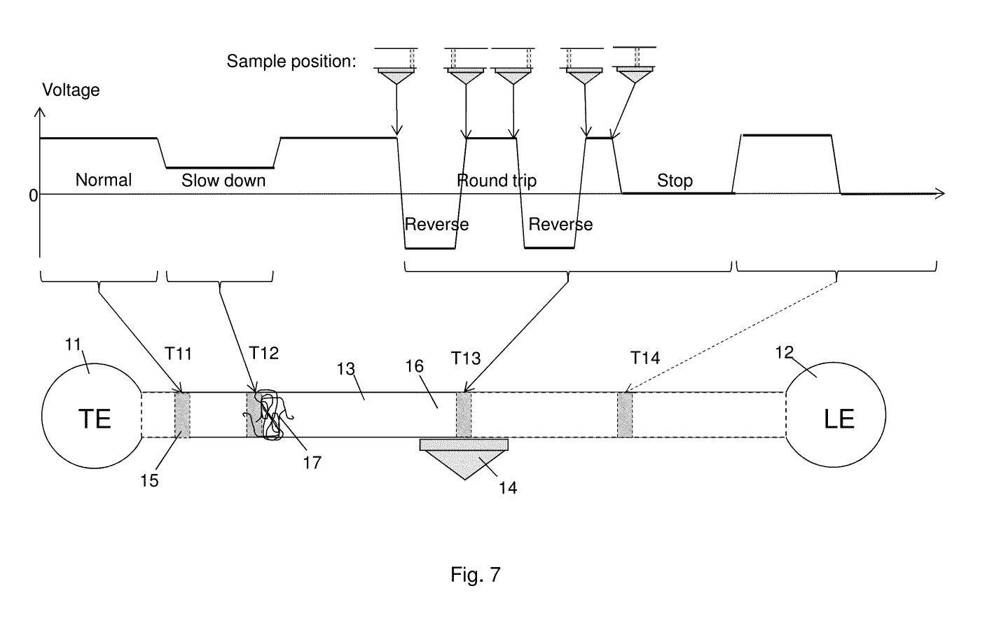

FIG. 7 illustrates an example of a voltage control sequence in a detection method using ITP equipped with a filter 17 and SPFS sensor 14 according to an embodiment of the present embodiment. During time period T11, when the sample band is located between the TE reservoir 11 and the filter 17, a normal voltage is applied to focus the sample 15. During time period T12, when the sample band is in the filter or its vicinity, the voltage is decreased and the sample moving speed is slowed down. During time period T13, the voltage is changed multiple times when the sample band is located within the sensor region 16. Within this time period, when the sample band first reaches the downstream edge of the SPFS sensor region 16 closer to the LE reservoir 12 (i.e. after the sample band has substantially passed through the sensor region), the voltage polarity is reversed and the sample moving direction is reversed. Then, when the sample reaches the upstream edge of the sensor region close to the TE reservoir (i.e. after the sample has substantially moved backward past the sensor region), the voltage is changed again to the original (normal) polarity. These “round trip” voltage changes can be done multiple times to cause the sample to make round trip movements in the sensor region. After the round trip movement, the sample is stopped in the center of the sensor region for a desired amount of time. During time period T14, the normal voltage is applied and the sample leaves the sensor region and moves towards the LE reservoir. At some time during this period (any time after the sample has left the sensor region), SPFS detection is conducted. During detection, the voltage can be turned off. As an alternative, a reverse voltage can be applied during the time period T14, to make the sample migrate towards the TE reservoir, as long as the sample is not in the sensor surface region when SPFS detection is conducted.

Using the above-described method, various analytes can be detected, including nucleic acids, proteins, metabolites, viruses, bacteria, cells, antibodies, etc. The mobility (μ) of the various components should satisfy μ_{LE}>μ_{target}, μ_{labeled probe}>μ_{TE}.

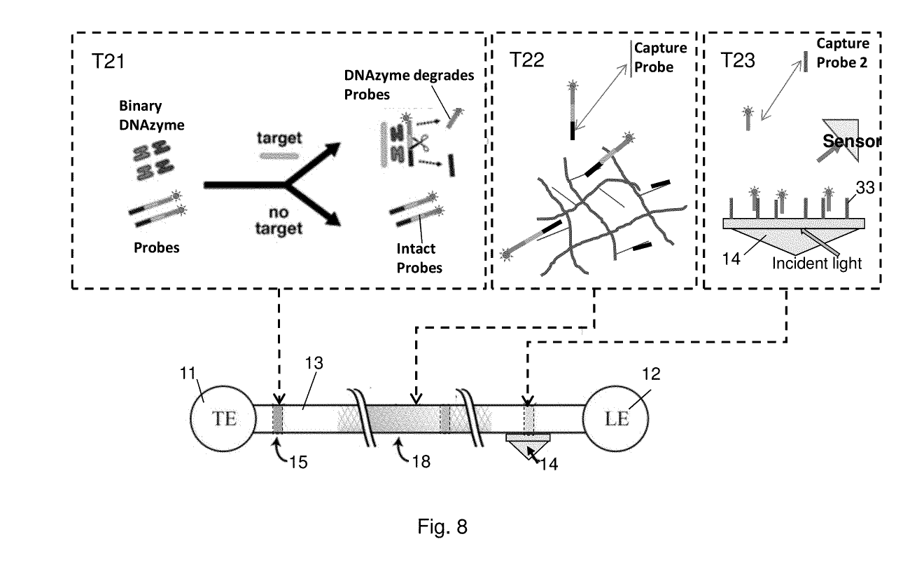

Further, DNAzyme amplification and separation mechanisms described in commonly-owned U.S. patent application Ser. No. 14/590,482, publication No. US 2015/0197791 (which is incorporated by reference herein) can be used in combination with SPFS techniques (see FIG. 8). US 2015/0197791 describes a “DNA detection method [which] combines DNAzyme reactions and on-chip isotachophoresis (ITP). A mixture of sample containing a target DNA and a DNAzyme sensor which is either (1) a catalytic molecular beacon or (2) a binary DNAzyme and a probe is loaded into a trailing electrolyte (TE) reservoir of a microfluidic chip. In the presence of the target DNA, the catalytic molecular beacon or the probe is cleaved to generate a fluorescent fragment. Enhanced DNAzyme reaction occurs at the TE-to-LE interface. Fluorescent signal from cleaved catalytic molecular beacon or probe is detected either at the location where DNAzyme reaction occurs or at a separate location. In the latter case, the microfluidic chip has a separation region containing a capture gel or a sieving matrix which allows the fluorescent fragment to pass through but captures or traps the uncleaved catalytic molecular beacon or probe.” (Id., Abstract.) This DNA detection method can be modified by incorporating an SPFS sensor in the microfluidic system in the manner shown in FIG. 8, so that the cleaved fragment which has a fluorescent tag can be captured on the SPFS sensor surface for detection.

More specifically, as shown in FIG. 8, during time period T21, DNAzyme reaction (enhanced hybridization) occurs in the focused sample 15 in a region (referred to as the DNAzyme reaction region) of the fluid channel 13 between the TE 11 and a capturing region 18. During time period T22, the sample 15 moves through the capturing region 18 where intact probes are captured by a matrix which has a capture probe immobilized on it. During time period T23, the sample 15 moves to the detection region above the surface of the SPFS sensor 14, where the degraded probes are detected.

Various modifications and improvements may be made to the above-described systems. As described in the Han et al. Lab on a Chip 2014 article “Increasing hybridization rate and sensitivity of DNA microarrays using isotachophoresis,” a narrow constriction can be equipped in the region upstream of the SPFS sensor, in order to make homogenous sample solution.

It is preferable to increase the sample volume to obtain higher signals. In the current ITP configuration, limitation of sample volume can be one of the challenges. One of the solutions can be to use a large sample reservoir.

The ITP chip shape is not necessarily straight. In order to avoid possible short circuit problem caused by SPFS gold sensor chip, other shape such as U-shape can be used.

It will be apparent to those skilled in the art that various modification and variations can be made in the detection method of the present invention without departing from the spirit or scope of the invention. Thus, it is intended that the present invention cover modifications and variations that come within the scope of the appended claims and their equivalents.

What is claimed is:

1. A microfluidic chip for detecting a biological analyte, comprising:
a fluid channel;
a first reservoir containing a low-mobility trailing electrolyte (TE) and connected to the fluid channel at a first location;
a second reservoir containing a high mobility leading electrolyte (LE) and connected to the fluid channel at a second location, wherein a voltage is applied between the first reservoir and the second reservoir; and
a SPFS (surface plasmon field enhanced fluorescence spectroscopy) sensor located at a detection region of the fluid channel, wherein the SPFS sensor has a metal surface which has capture molecules immobilized on it and which forms a part of an inner surface of the fluid channel.

2. A method for detecting a target analyte, comprising:
providing a microfluidic chip having a fluid channel, a first reservoir containing a low-mobility trailing electrolyte (TE) and connected to the fluid channel at a first location, a second reservoir containing a high mobility leading electrolyte (LE) and connected to the fluid channel at a second location, and a SPFS (surface plasmon field enhanced fluorescence spectroscopy) sensor at a detection region of the fluid channel, wherein the SPFS sensor has a metal surface which has capture molecules immobilized on it and which forms a part of an inner surface of the fluid channel;
loading the target analyte and a fluorescent labeled probe into the first reservoir of the microfluidic chip, wherein the target analyte and the fluorescent labeled probe are capable of binding to each other to form a complex, and wherein the complex is capable of binding to the capture molecules on the surface of the SPFS sensor;
applying a voltage between the first and second reservoirs; and
detecting a fluorescent signal in the detection region.
